(19) United States
(12) Patent Application Publication
(10) Pub. No.: US 2026/0112497 A1
(43) Pub. Date: Apr. 23, 2026

(54) RISK ESTIMATION DEVICE, RISK ESTIMATION METHOD, AND RECORDING MEDIUM

(71) Applicant: NEC Corporation, Tokyo (JP)

(72) Inventors: Chenhui HUANG, Tokyo (JP); Kensuke WAGATA, Tokyo (JP); Fumiyuki NIHEY, Tokyo (JP)

(73) Assignee: NEC Corporation, Tokyo (JP)

(21) Appl. No.: 19/350,429

(22) Filed: Oct. 6, 2025

(30) Foreign Application Priority Data
Oct. 22, 2024 (JP) 2024-185806

(51) Int. Cl. G16H 50/30; G06N 7/01; G06N 20/00

(52) U.S. Cl. CPC G16H 50/30; G06N 7/01; G06N 20/00

(57) ABSTRACT

In the risk estimation device, the acquisition means acquires data of a plurality of different modalities. The encoder converts data of each modality into data indicating a probability distribution in a latent space. The predictor predicts a risk corresponding to each modality based on the probability distribution. The calculation means integrates the risks corresponding to the respective modalities by using weights corresponding to the respective modalities to calculate an estimation result. By using the risk estimation device to estimate disease risk, it is possible to support decision making regarding the subject's lifestyle.

### INCORPORATION BY REFERENCE

This application is based upon and claims the benefit of priority from Japanese Patent Application 2024-185806, filed on Oct. 22, 2024, the disclosure of which is incorporated herein in its entirety by reference.

### TECHNICAL FIELD

The present disclosure relates to risk estimation.

### BACKGROUND ART

A disease risk estimation technique using a machine learning model is known. For example, Patent Document 1 describes a multi-modal machine learning model that predicts the progression of dementia using a plurality of types of input data. In Patent Document 1, the final prediction result is generated by integrating prediction results based on a plurality of pieces of input data, according to a prediction interval from a reference time point to a future time point being predicted.
Patent Document 1: International Publication WO 2023/276976

### SUMMARY

However, in the method of Patent Document 1, since prediction results based on a plurality of pieces of input data are integrated according to the prediction interval, a highly accurate prediction result is not necessarily obtained.

One object of the present disclosure is to provide a risk estimation device capable of highly accurate risk estimation.

According to an example aspect of the present invention, there is provided a risk estimation device comprising:
an acquisition means configured to acquire data of a plurality of different modalities;
an encoder configured to convert data of each modality into data indicating a probability distribution in a latent space;
a predictor configured to predict a risk corresponding to each modality based on the probability distribution; and
a calculation means configured to calculate an estimation result by integrating the risks corresponding to the respective modalities using weights corresponding to the respective modalities.

According to another example aspect of the present invention, there is provided a risk estimation method executed by a computer, comprising:
acquiring data of a plurality of different modalities;
converting data of each modality into data indicating a probability distribution in a latent space;
predicting a risk corresponding to each modality based on the probability distribution; and
calculating an estimation result by integrating the risks corresponding to the respective modalities using weights corresponding to the respective modalities.

According to still another example aspect of the present invention, there is provided a program that causes a computer to execute processing comprising:
acquiring data of a plurality of different modalities;
converting data of each modality into data indicating a probability distribution in a latent space;
predicting a risk corresponding to each modality based on the probability distribution; and
calculating an estimation result by integrating the risks corresponding to the respective modalities using weights corresponding to the respective modalities.

### Effect

According to the present disclosure, highly accurate risk estimation can be achieved.

### BRIEF DESCRIPTION OF THE DRAWINGS

FIG. 1 illustrates an overall configuration of a risk estimation device according to the present disclosure;

FIG. 2 is a block diagram illustrating a hardware configuration of the risk estimation device;

FIG. 3 is a block diagram illustrating a functional configuration of a risk estimation model training device;

FIG. 4 is a flowchart of training processing;

FIG. 5 is a block diagram illustrating a functional configuration of the risk estimation device;

FIG. 6 is a flowchart of the risk estimation processing;

FIG. 7 is a block diagram illustrating another functional configuration of the risk estimation device;

FIG. 8 is a block diagram illustrating another functional configuration of the risk estimation device; and

FIG. 9 is a flowchart of another risk estimation processing.

### EXAMPLE EMBODIMENTS

Hereinafter, preferred example embodiments of the present disclosure will be described with reference to the drawings.

### First Example Embodiment

### [Overall Configuration]

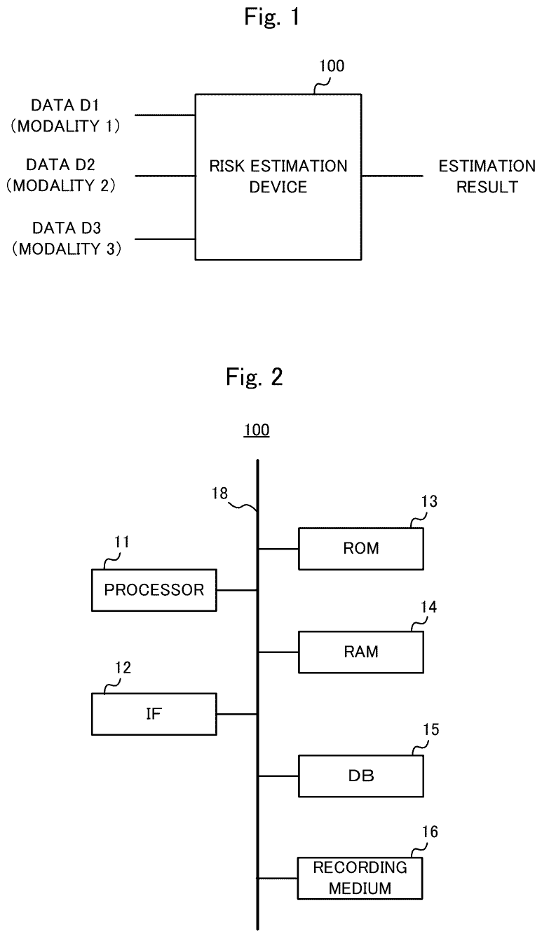

FIG. 1 illustrates an overall configuration of a risk estimation device according to the present disclosure. The risk estimation device 100 estimates a disease risk of a subject based on health data of the subject. Specifically, multimodal data, that is, data of a plurality of different modalities is input to the risk estimation device 100. Note that the term modality means a method, means, or the like for expressing information, and the term multimodal data means pieces of data in different data formats such as text, image, audio, and sensor data. In the present example embodiment, the multimodal data includes, for example, various pieces of data obtained by health check or the like, such as height, weight, sex, blood pressure, body mass index (BMI), body fat percentage, neutral fat value, smoking status and amount, drinking status and amount, and the like of the subject.

As illustrated in FIG. 1, a plurality of pieces of data (in this example, pieces of data D1 to D3) of different modalities are input to the risk estimation device 100. The risk estimation device 100 predicts a disease risk based on the input data of each modality, integrates the prediction results of the modalities, and outputs a final estimation result. At this time, the risk estimation device 100 converts the data of each modality into a probability distribution in a latent space, and integrates the prediction results of the modalities according to the similarity between the obtained probability distribution and a predetermined reference distribution. As a result, the risk estimation device 100 can integrate the prediction results of the modalities at an appropriate ratio according to the characteristics of the data of each modality, and can estimate the disease risk with high accuracy.

The risk estimation device 100 can be suitably applied in the medical or healthcare field. For example, the risk estimation device 100 can be used to estimate the risk of a lifestyle-related disease based on data obtained in a regular health check.

### [Hardware Configuration]

FIG. 2 is a block diagram illustrating a hardware configuration of the risk estimation device 100. As illustrated in the drawing, the risk estimation device 100 includes a processor 11, an interface (IF) 12, a read only memory (ROM) 13, a random access memory (RAM) 14, a database (DB) 15, and a recording medium 16. The components are connected via a bus 18, for example.

The processor 11 is a computer such as a central processing unit (CPU), and controls the risk estimation device 100 by executing a program prepared in advance. As the processor 11, a CPU, a graphics processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination of these can be used.

In addition, the processor 11 loads a program stored in the ROM 13 or the recording medium 16 into the RAM 14 and executes each process coded in the program. The processor 11 functions as part or all of the risk estimation device 100. Specifically, the processor 11 executes training processing and risk estimation processing to be described later.

The IF 12 transmits and receives data to and from an external device. Specifically, in the training phase, the risk estimation device 100 receives multimodal data on a plurality of persons as training data through the IF 12. Furthermore, in the estimation phase, that is, at the time of risk estimation, the risk estimation device 100 receives the multimodal data of the subject through the IF 12 and outputs an estimation result of the disease risk to the display device or another external device.

The ROM 13 stores various programs executed by the processor 11. The RAM 14 is used as a working memory during execution of various types of processing by the processor 11.

The DB 15 stores various algorithms, data, machine learning models, and the like used when the risk estimation device 100 executes the training processing and the risk estimation processing to be described later.

The recording medium 16 is a non-volatile and non-transitory storage medium such as a disk-shaped recording medium or a semiconductor memory. The recording medium 16 may be configured to be attachable to and detachable from the risk estimation device 100. The recording medium 16 records various programs executed by the processor 11.

In addition to the above, the risk estimation device 100 may include a display device such as a liquid crystal display and an input device such as a keyboard and a mouse. The display and input devices are used by an operator of the risk estimation device 100, for example.

### [Training Phase]

Next, the training phase of the risk estimation model will be described.

### (Training Device)

The risk estimation device 100 estimates the disease risk using a trained risk estimation model. Note that, in the following description, the risk estimation model estimates the disease risk from the pieces of data D1 to D3 of three different modalities as an example, but the number of types of data constituting the multimodal data is not limited thereto.

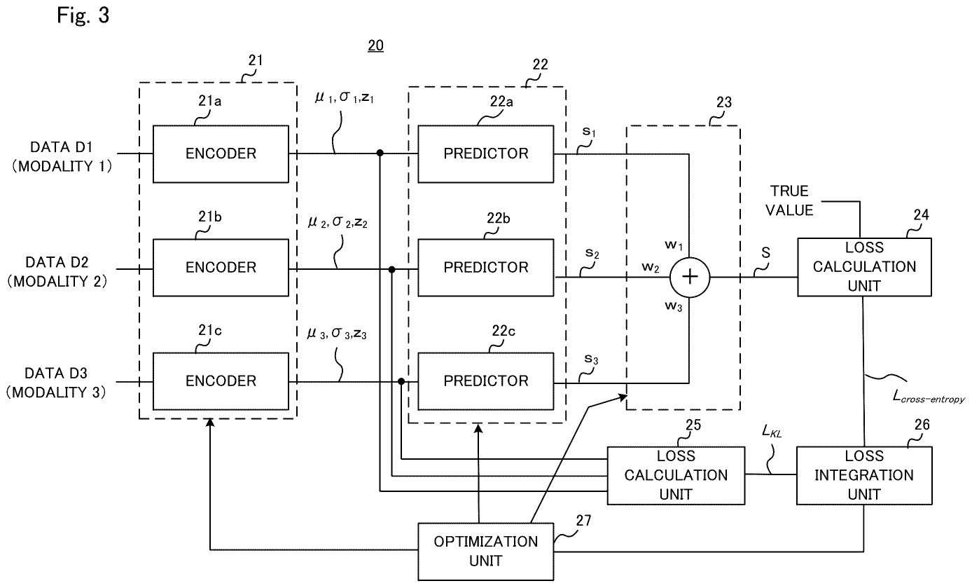

FIG. 3 is a block diagram illustrating a functional configuration of a risk estimation model training device 20. The training device 20 includes an encoder unit 21, a prediction unit 22, an integration unit 23, loss calculation units 24 and 25, a loss integration unit 26, and an optimization unit 27. The encoder unit 21 includes encoders 21a to 21c corresponding to modalities 1 to 3. The prediction unit 22 includes predictors 22a to 22c corresponding to the modalities 1 to 3.

The risk estimation model includes the encoder unit 21, the prediction unit 22, and the integration unit 23. Specifically, a neural network forms the encoder unit 21 and the prediction unit 22. In the training phase, the training device 20 generates a trained risk estimation model by optimizing the neural network using training data and optimizing the weights used by the integration unit 23.

As the training data, multimodal disease risk data for a plurality of persons is prepared. Specifically, the training data is obtained by collecting attribute data and disease risk values for a plurality of persons. As the attribute data, for example, those having high relevance to the disease risk to be estimated among height, weight, sex, blood pressure, BMI, neutral fat value, blood glucose level, smoking status and amount, drinking status and amount, and the like are used. Note that the disease risk value of each individual corresponds to the correct data in so-called supervised learning, and is hereinafter also referred to as a “true value”. For example, it is assumed that the risk of heart disease is estimated as the disease risk using the blood pressure, BMI, and neutral fat value as the pieces of data D1 to D3. In this case, as the training data, for a plurality of persons, data including blood pressure, BMI, and neutral fat value as the input data and the presence or absence of heart disease as the true value is collected.

In FIG. 3, the pieces of data D1 to D3 of the respective modalities 1 to 3 are input to the encoder unit 21. The data D1 is input to the encoder 21a, the data D2 is input to the encoder 21b, and the data D3 is input to the encoder 21c. Each of the encoders 21a to 21c projects the input data to a latent space. The “latent space” is an abstract space for expressing information included in the original data in fewer dimensions, and in the latent space, essential features and patterns of data are expressed in fewer dimensions. The expression “projects to a latent space” refers to converting the original data into points on the latent space, which is also referred to as “mapping to the latent space”.

Next, each of the encoders 21a to 21c calculates a probability distribution in the latent space for the one of the pieces of input data D1 to D3 of the corresponding modality, and outputs probability distribution data indicating the probability distribution. Specifically, the probability distribution data includes an average μ, a standard deviation σ, and a latent representation z. The latent representation z is expressed by the following Formula (1), and is also referred to as an intermediate representation, a hidden representation, a latent variable, or the like.

$$z = μ + σ ε \qquad ( 1 )$$

$$ε \sim N ( 0 , 1 ) \qquad ( N is Standard Normal Distribution )$$

The probability distribution data output from each of the encoders 21a to 21c is input to the one of the predictors 22a to 22c of the corresponding modality. Further, the probability distribution data output from each of the encoders 21a to 21c is input to the loss calculation unit 25.

The predictors 22a to 22c calculate disease risk scores (hereinafter referred to as “risk scores”) s₁ to s₃, each corresponding to the data of the corresponding modality, based on the input latent representation z, and output the scores s₁ to s₃ to the integration unit 23.

The integration unit 23 calculates an integrated risk score S by weighting and adding the risk scores s₁ to s₃ of the respective modalities. Specifically, assuming that the weights of the modalities 1 to 3 are w₁ to w₃, respectively, the integration unit 23 calculates the integrated risk score S by the following Formula (2) and outputs the integrated risk score S to the loss calculation unit 24.

$$S = {w}_{1} \times {s}_{1} + {w}_{2} \times {s}_{2} + {w}_{3} \times {s}_{3} \qquad ( 2 )$$

The loss calculation unit 24 outputs a cross entropy loss L_{cross-entropy} of the integrated risk score S and the true values corresponding to the respective pieces of input data D₁ to D₃ to the loss integration unit 26.

On the other hand, the loss calculation unit 25 calculates the similarity between the probability distribution of each modality and a reference distribution using the probability distribution data input from each of the encoders 21a to 21c. In a case where the input data D is real number data, a normal distribution is used as the reference distribution. Therefore, the loss calculation unit 25 calculates the Kullback-Leibler (KL) divergence between the probability distribution of each modality and the normal distribution N(0,1) as a loss L_{KL} by the following Formula (3) using the average u and the standard deviation σ of each modality.

$${L}_{KL} = {D}_{KL} [ f ( N ( {μ}_{i} , {σ}_{i} ) ‖ f ( N ( 0 , 1 ) ) ] \qquad ( 3 )$$

Note that, in a case where the input data is not real data, the loss calculation unit 25 can use a log-normal distribution, a Poisson distribution, a multinomial logit, an ordinal logit, or the like as the reference distribution according to the format of the input data D.

The loss integration unit 26 calculates a weighted sum of the loss L_{KL} and the loss L_{cross-entropy} by the following Formula (4), and outputs the weighted sum to the optimization unit 27 as a total loss Lₜₒₜₐₗ.

$${L}_{tot al} = {L}_{KL} + λ {L}_{cross - entropy} \qquad ( 4 )$$

Note that “λ” indicates a weight for weighted addition of the first and second losses.

The optimization unit 27 optimizes the encoder unit 21 and the prediction unit 22 based on the total loss Lₜₒₜₐₗ, and optimizes the weights w₁ to w₃ used by the integration unit 23. Specifically, the optimization unit 27 optimizes the parameters of the neural network forming the encoder unit 21 and the prediction unit 22 and optimizes the weights w₁ to w₃ used by the integration unit 23, so as to reduce the total loss Lₜₒₜₐₗ. Here, since the total loss Lₜₒₜₐₗ is a weighted sum of the loss L_{KL} and the loss L_{cross-entropy}, the optimization unit 27 performs optimization so that the KL divergence between the probability distribution output by each of the encoders 21a to 21c and the reference distribution becomes small, that is, the similarity between the probability distribution and the reference distribution becomes high. At the same time, the optimization unit 27 performs optimization so as to reduce the error between the integrated risk score S output by the integration unit 23 and the true value.

According to the above optimization, since the risk score s of the modality whose probability distribution has a high degree of similarity to the reference distribution has a high reliability, it is reflected in the integrated risk score S with a large weight. In addition, since the risk score s of the modality whose probability distribution has a low degree of similarity to the reference distribution has a low reliability, and it is reflected in the integrated risk score S with a small weight. In this way, the trained risk estimation model can calculate the integrated risk score S using the appropriate weights according to the characteristics of the modalities of the input data.

### (Training Processing)

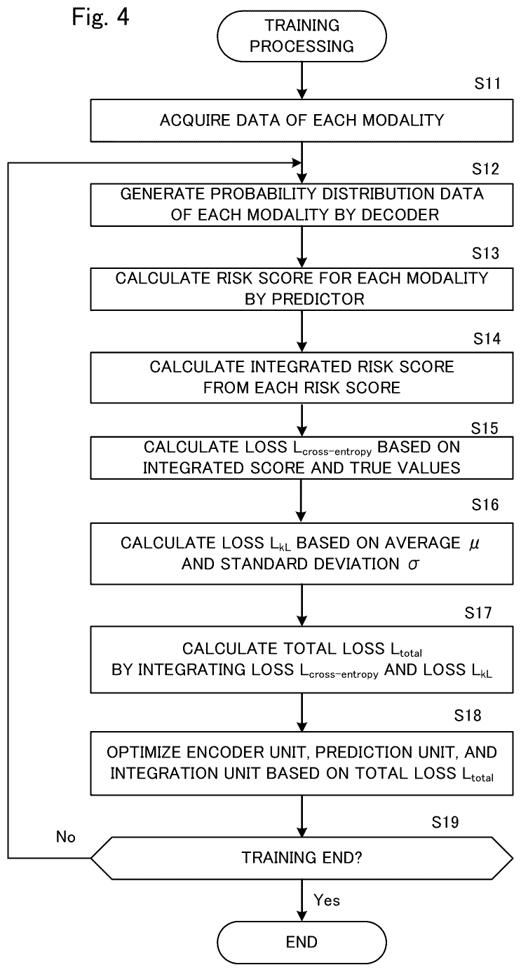

Next, the training processing performed by the above training device 20 will be described. FIG. 4 is a flowchart of the training processing. This processing is achieved by the processor 11 illustrated in FIG. 2 executing a program prepared in advance and operating as each component illustrated in FIG. 3.

First, the encoder unit 21 acquires data of each modality included in the training data (step S11). Next, the encoder unit 21 projects each data to a latent space by the corresponding one of the encoders 21a to 21c to generate probability distribution data (step S12). Next, the prediction unit 22 calculates risk scores s₁ to s₃ of the respective modalities by the respective predictors 22a to 22c (step S13). Next, the integration unit 23 integrates the risk scores s₁ to s₃ of the respective modalities using the weights w₁ to w₃ to calculate the integrated risk score S (step S14).

Next, the loss calculation unit 24 calculates the loss L_{cross-entropy} based on the integrated risk score S and the true values (step S15). In addition, the loss calculation unit 25 calculates the loss L_{KL} using the average u and the standard deviation σ of each modality (step S16). Next, the loss integration unit 26 calculates the total loss Lₜₒₜₐₗ from the loss L_{cross-entropy} and the loss L_{KL} (step S17). Next, the optimization unit 27 optimizes the parameters of the encoder unit 21 and the prediction unit 22 and the weights w₁ to w₃ of the integration unit 23 based on the total loss Lₜₒₜₐₗ (step S18).

Next, the training device 20 determines whether a predetermined training end condition has been satisfied (step S19). Examples of the training end condition include that a predetermined number of pieces of attribute data prepared as training data has been used, the total loss has become equal to or less than a predetermined value, and the total loss has converged. If the training end condition is not satisfied (step S19: No), the process returns to step S12. On the other hand, if the training end condition is satisfied (step S19: Yes), the training processing ends.

### [Estimation Phase]

Next, the estimation phase by the risk estimation device will be described. In the estimation phase, the risk estimation device 100 estimates the disease risk of a certain subject based on multimodal data of the subject. At this time, the risk estimation device 100 uses the risk estimation model trained in the training phase, specifically, the encoder unit 21, the prediction unit 22, and the integration unit 23.

### (Risk Estimation Device)

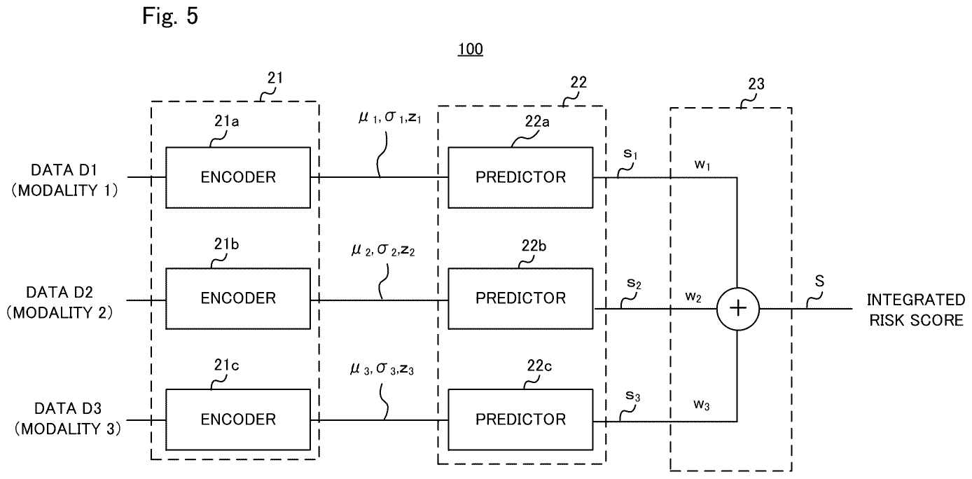

FIG. 5 is a block diagram illustrating a functional configuration of the risk estimation device. The risk estimation device 100 includes the encoder unit 21, the prediction unit 22, and the integration unit 23 optimized in the training phase.

Pieces of data D1 to D3 of three different modalities are input to the encoder unit 21 for a certain subject. Each of the encoders 21a to 21c projects the pieces of input data D1 to D3 to a latent space, generates probability distribution data including the average μ, the standard deviation σ, and the latent representation z, and outputs the probability distribution data to each of the predictors 22a to 22c.

The predictors 22a to 22c calculate the risk scores s₁ to s₃ of the respective modalities based on the input latent representation z, and output the scores to the integration unit 23. The integration unit 23 performs weighted addition of the risk scores s₁ to s₃ of the respective modalities using the weights w₁ to w₃ optimized in the training phase, and outputs the integrated risk score S. In this way, the disease risk for a specific subject can be predicted using the trained risk estimation model.

### (Risk Estimation Processing)

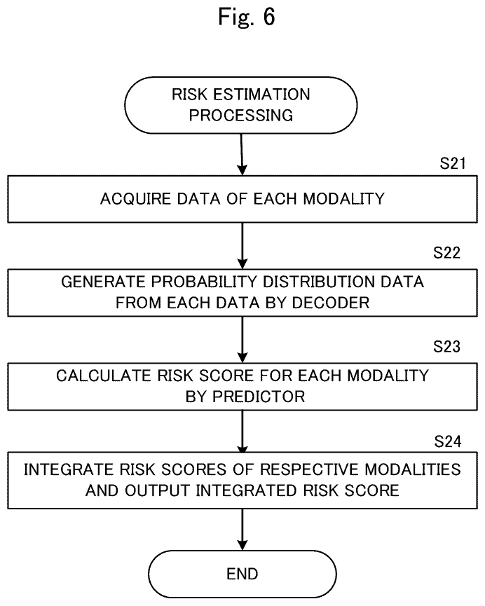

Next, risk estimation processing executed by the risk estimation device 100 will be described. FIG. 6 is a flowchart of the risk estimation processing. This processing is achieved by the processor 11 illustrated in FIG. 2 executing a program prepared in advance and operating as each element illustrated in FIG. 5.

First, the encoder unit 21 acquires the pieces of data D1 to D3 of the respective modalities for the subject (step S21). Next, the encoder unit 21 generates probability distribution data from the pieces of data D1 to D3 (step S22). Next, the prediction unit 22 calculates the risk scores s₁ to s₃ of the respective modalities using the probability distribution data (step S23). Next, the integration unit 23 calculates the integrated risk score S from the risk scores s₁ to s₃ of the respective modalities using the weights w₁ to w₃ optimized in the training phase, and outputs the integrated risk score S (step S24). Then, the risk estimation processing ends.

### MODIFICATION

Next, modification examples of the above example embodiment will be described. The following modifications can be appropriately combined and applied to the above example embodiment.

### First Modification

In the above example embodiment, in the training phase, the weights w (w₁ to w₃) of the integration unit 23 are determined using data of a plurality of persons. On the other hand, in the inference phase, since the disease risk is estimated using the data of the subject, the weights w determined in the training phase are not necessarily optimal for the subject. For example, for a certain subject, there may be individual circumstances such as the reliability of data of a certain modality being low. For example, a subject X has unstable blood pressure measurement data and thus its reliability is low.

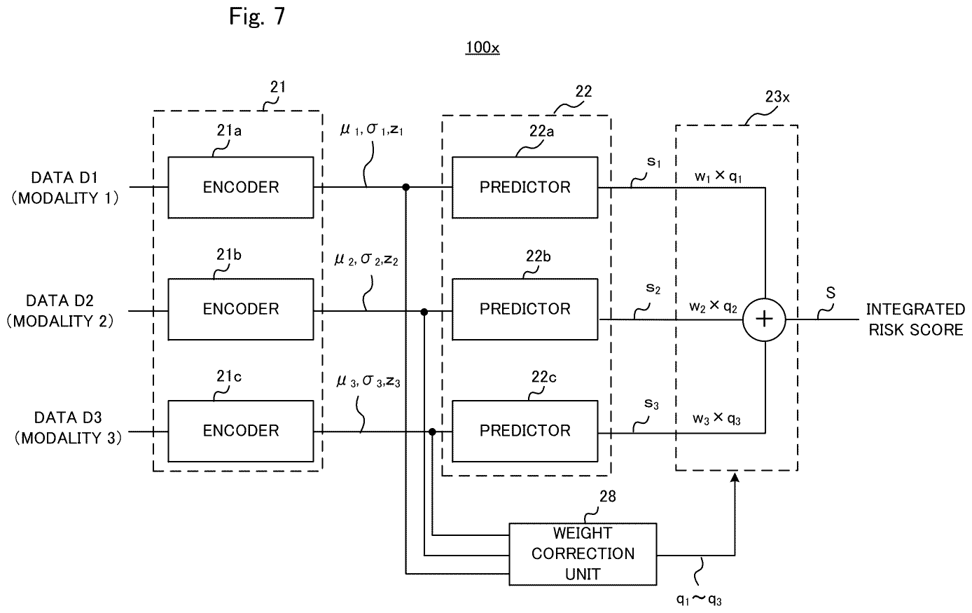

From such a perspective, in the first modification, the weights w used by the integration unit 23 can be corrected in the inference phase. FIG. 7 illustrates a functional configuration of a risk estimation device 100x according to the first modification. As can be understood by comparing it with FIG. 5, the risk estimation device 100x according to the first modification includes a weight correction unit 28 in addition to the configuration of FIG. 5.

Probability distribution data is input from each of the encoders 21a to 21c to the weight correction unit 28. The weight correction unit 28 corrects the weight w (w₁ to w₃) used by the integration unit 23 based on the similarities between the probability distributions obtained based on the pieces of data D1 to D3 input to the encoders 21a to 21c in the inference phase and the reference distributions. Specifically, the weight correction unit 28 calculates KL divergence between the probability distribution obtained from each of the pieces of data D1 to D₃ and the reference distribution, and determines a correction coefficient qᵢ based on the obtained KL divergence.

Specifically, the weight correction unit 28 determines the correction coefficient qᵢ by the following Formula.

$${q}_{i} = \{ \begin{matrix}0 : & {D}_{KL} [ f ( N ( {μ}_{i} , {σ}_{i} ) ‖ f ( N ( 0 , 1 ) ) ] > {t}_{KL} \\ 1 : & {D}_{KL} [ f ( N ( {μ}_{i} , {σ}_{i} ) ‖ f ( N ( 0 , 1 ) ) ] ¬ {t}_{KL}\end{matrix} \qquad ( 5 )$$

Note that “t_{KL}” is a predetermined threshold value.

That is, in a case where the KL divergence of a certain modality i is larger than a threshold t_{KL}, the weight correction unit 28 sets the correction coefficient qᵢ to “0”. As a result, in a case where the similarity between the probability distribution of the modality i and the reference distribution is low, the risk score of the modality is considered to have low reliability and is not reflected in the integrated risk score. On the other hand, in a case where the KL divergence of a certain modality i is equal to or smaller than the threshold t_{KL}, the weight correction unit 28 sets the correction coefficient qᵢ to “1”. As a result, in a case where the similarity between the probability distribution of the modality i and the reference distribution is high, the risk score of the modality is reflected in the integrated risk score at the ratio determined in the training phase. As described above, by correcting the weights w of the integration unit 23 based on the data actually input in the inference phase, it is possible to estimate the disease risk according to the personal characteristics of the subject and the like.

Note that the value of the threshold value t_{KL} may be the same value for all modalities, or may be a different value for each modality. In addition, the value of the threshold t_{KL} may be the same value for all subjects in the disease risk estimation, or may be a different value for each subject.

### Second Modification

In the first example embodiment described above, the risk estimation device is applied to generate attribute data on human health, but the application of the present disclosure is not limited thereto. For example, the present disclosure may be applied to inspection and diagnosis of machines and devices. That is, the method of the present disclosure may be applied to estimate the state of the machine or device based on data of a plurality of modalities detected and collected in inspection or diagnosis.

### Second Example Embodiment

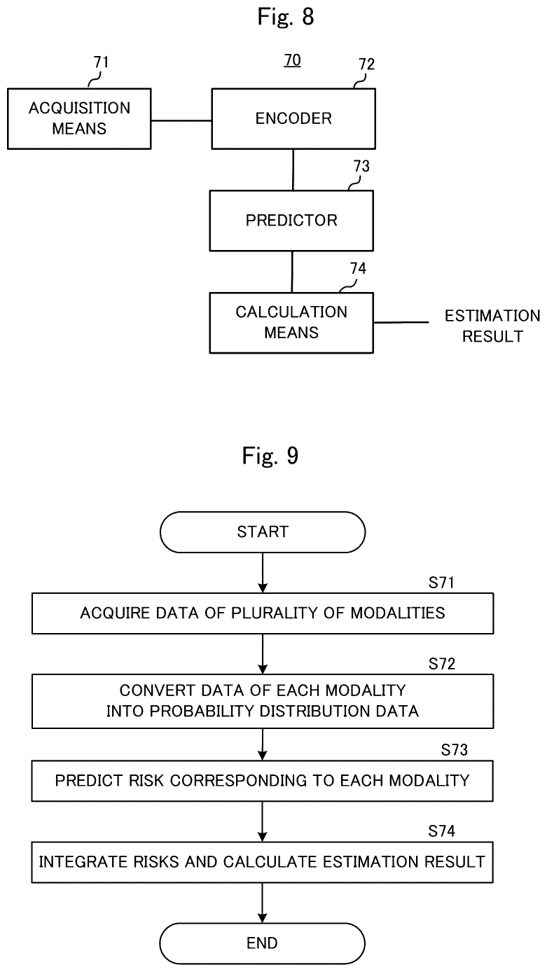

FIG. 8 is a block diagram illustrating a functional configuration of a risk estimation device of a second example embodiment. A risk estimation device 70 includes an acquisition means 71, an encoder 72, a predictor 73, and a calculation means 74.

FIG. 9 is a flowchart of processing by the risk estimation device according to the second example embodiment. The acquisition means 71 acquires data of a plurality of different modalities (step S71). The encoder 72 converts data of each modality into data indicating a probability distribution in a latent space (step S72). The predictor 73 predicts a risk corresponding to each modality based on the probability distribution (step S73). The calculation means 74 integrates the risks corresponding to the respective modalities by using weights corresponding to the respective modalities to calculate an estimation result (step S74).

According to the risk estimation device 70 of the second example embodiment, the risk can be estimated with high accuracy.

A part or all of the example embodiments described above may also be described as the following supplementary notes, but not limited thereto.

### Supplementary Note 1

A risk estimation device comprising:
an acquisition means configured to acquire data of a plurality of different modalities;
an encoder configured to convert data of each modality into data indicating a probability distribution in a latent space;
a predictor configured to predict a risk corresponding to each modality based on the probability distribution; and
a calculation means configured to calculate an estimation result by integrating the risks corresponding to the respective modalities using weights corresponding to the respective modalities.

### Supplementary Note 2

The risk estimation device according to Supplementary note 1, further comprising an optimization means configured to optimize the weights corresponding to the respective modalities based on similarity between the probability distribution corresponding to each of the modalities and a predetermined reference distribution.

### Supplementary Note 3

The risk estimation device according to Supplementary note 2, wherein the optimization means sets the weight to a larger value as the similarity between the probability distribution and the reference distribution is higher, and sets the weight to a smaller value as the similarity between the probability distribution and the reference distribution is lower.

### Supplementary note 4

The risk estimation device according to Supplementary note 2,
wherein data indicating the probability distribution includes an average and a standard deviation, and
wherein the similarity is indicated by KL divergence between the probability distribution and the reference distribution.

### Supplementary Note 5

The risk estimation device according to Supplementary note 1, further comprising a weight correction means configured to correct the weights corresponding to the respective modalities based on the probability distributions corresponding to the respective modalities.

### Supplementary Note 6

The risk estimation device according to Supplementary note 5, wherein the weight correction means calculates correction coefficients for correcting the weights corresponding to the respective modalities based on similarities between probability distributions of the respective modalities and reference distributions.

### Supplementary Note 7

The risk estimation device according to Supplementary note 6, wherein the weight correction means sets the correction coefficient to 0 when the similarity is greater than a predetermined threshold, and sets the correction coefficient to 1 when the similarity is equal to or less than the predetermined threshold.

### Supplementary Note 8

The risk estimation device according to Supplementary note 1, wherein the predictor predicts a disease risk of a subject based on data of a plurality of modalities related to health of the subject by a trained machine learning model.

### Supplementary Note 9

A risk estimation method executed by a computer, comprising:
acquiring data of a plurality of different modalities;
converting data of each modality into data indicating a probability distribution in a latent space;
predicting a risk corresponding to each modality based on the probability distribution; and
calculating an estimation result by integrating the risks corresponding to the respective modalities using weights corresponding to the respective modalities.

### Supplementary Note 10

A program that causes a computer to execute processing comprising:
acquiring data of a plurality of different modalities;
converting data of each modality into data indicating a probability distribution in a latent space;
predicting a risk corresponding to each modality based on the probability distribution; and
calculating an estimation result by integrating the risks corresponding to the respective modalities using weights corresponding to the respective modalities.

While the present disclosure has been described with reference to the example embodiments and examples, the present disclosure is not limited to the above example embodiments and examples. Various changes which can be understood by those skilled in the art within the scope of the present disclosure can be made in the configuration and details of the present disclosure.

### DESCRIPTION OF SYMBOLS

11 Processor
20 Training device
21 Encoder unit
21a-21c encoder
22 Prediction unit
22a-22c Predictor
23 Integration unit
24, 25 Loss calculation unit
26 Loss integration unit
27 Optimization unit
28 Weight correction unit
100, 100x Risk estimation device

1. A risk estimation device comprising:
at least one memory configured to store instructions; and
at least one processor configured to execute the instructions to:
acquire data of a plurality of different modalities;
convert data of each modality into data indicating a probability distribution in a latent space;
predict a risk corresponding to each modality based on the probability distribution; and
calculate an estimation result by integrating the risks corresponding to the respective modalities using weights corresponding to the respective modalities.

2. The risk estimation device according to claim 1, wherein the processor is further configured to execute the instructions to optimize the weights corresponding to the respective modalities based on similarity between the probability distribution corresponding to each of the modalities and a predetermined reference distribution.

3. The risk estimation device according to claim 2, wherein the processor sets the weight to a larger value as the similarity between the probability distribution and the reference distribution is higher, and sets the weight to a smaller value as the similarity between the probability distribution and the reference distribution is lower.

4. The risk estimation device according to claim 2,
wherein data indicating the probability distribution includes an average and a standard deviation, and
wherein the similarity is indicated by KL divergence between the probability distribution and the reference distribution.

5. The risk estimation device according to claim 1, wherein the processor is further configured to execute the instructions to correct the weights corresponding to the respective modalities based on the probability distributions corresponding to the respective modalities.

6. The risk estimation device according to claim 5, wherein the processor calculates correction coefficients for correcting the weights corresponding to the respective modalities based on similarities between probability distributions of the respective modalities and reference distributions.

7. The risk estimation device according to claim 6, wherein the processor sets the correction coefficient to 0 when the similarity is greater than a predetermined threshold, and sets the correction coefficient to 1 when the similarity is equal to or less than the predetermined threshold.

8. The risk estimation device according to claim 1, wherein the processor predicts a disease risk of a subject based on data of a plurality of modalities related to health of the subject by a trained machine learning model.

9. A risk estimation method executed by a computer, comprising:
acquiring data of a plurality of different modalities;
converting data of each modality into data indicating a probability distribution in a latent space;
predicting a risk corresponding to each modality based on the probability distribution; and
calculating an estimation result by integrating the risks corresponding to the respective modalities using weights corresponding to the respective modalities.

10. A non-transitory computer-readable medium storing a program, the program causing a computer to execute processing comprising:
acquiring data of a plurality of different modalities;
converting data of each modality into data indicating a probability distribution in a latent space;
predicting a risk corresponding to each modality based on the probability distribution; and
calculating an estimation result by integrating the risks corresponding to the respective modalities using weights corresponding to the respective modalities.
